(19) United States
(12) Patent Application Publication
(10) Pub. No.: US 2014/0379005 A1
(43) Pub. Date: Dec. 25, 2014

(54) Monolithic Three-Dimensional Prosthesis for the Treatment of Hernias and Manufacturing Method

(71) Applicant: Herniamesh S.R.L., Chivasso (IT)

(72) Inventors: Ermanno E. TRABUCCO, Muttontown, NY (US); Pier Aldo CREPALDI, Vercelli (IT)

(73) Assignee: Herniamesh S.R.L., Chivasso (IT)

(21) Appl. No.: 14/310,335

(22) Filed: Jun. 20, 2014

(30) Foreign Application Priority Data
Jun. 21, 2013 (IT) TO2013A000511

(51) Int. Cl. A61F 2/00; B29C 51/28

(52) U.S. Cl. CPC A61F 2/0063; B29C 51/28; A61F 2002/0068; B29K 2995/0056; B29K 2101/12
USPC 606/151; 264/314; 264/294

(57) ABSTRACT

Prosthesis to treat hernia, can include a single mesh sheet having biocompatible fibers. It has a flat portion, an unstiffened protuberance formed from the flat portion, and an opening in the flat portion formed by the protuberance having a first diameter. The protuberance includes a proximal portion closest to the flat portion having a proximal portion height, and a distal portion disposed farthest from the flat portion having a distal portion height, a second diameter, and an end of the distal portion is unstiffened. The proximal portion height is a distance from the flat portion to a beginning of the distal portion, and the distal portion height can be a distance from the flat portion to the end of the distal portion. The second diameter is greater than the first diameter, and a first ratio of the distal portion height to the proximal portion height is approximately 1.4 to 3.0.

### CROSS-REFERENCE TO RELATED APPLICATIONS

The present application claims priority to Italian Application No. TO2013A000511 filed Jun. 21, 2013. This application is incorporated herein in its entirety.

### FIELD OF THE INVENTION

The invention relates generally to a prosthesis for the surgical treatment of hernias, including indirect and recurrent inguinal and femoral hernias.

### BACKGROUND

Three dimensional prostheses are typically formed from a network of biocompatible material (meshes) having a flat portion and a protrusion portion protruding therefrom. The flat portion can reinforce the rear of the inguinal canal and avoid the risk of a recurrence of the hernia. The protuberance extends into the cavity left by the reduction of the hernia.

Prior art prostheses by one of the inventors of the present invention, U.S. Publication No. 2005/0021058 to Negro, detail a mesh protuberance which has a uniform diameter. Here, the protuberance extends into the cavity left by the reduction of the hernia but is not anchored or reinforced on the rear of the cavity.

Another example is U.S. Pat. No. 6,241,768 to Agarwal et al., which can have a large end to the protuberance, opposite the flat portion, making it difficult to place the protuberance through the cavity and extend the large end. This structure adds to the amount of time and effort it takes to properly place the prostheses during the surgical procedure. The previously known implants that have a relatively large mass form a relatively large foreign body in the patient, which is not advantageous for the healing process. Further, Agarwal's prosthesis is not monolithic, it is typically formed from two mesh sheets.

Other prior art, for example U.S. Pat. No. 7,156,858 to Schuldt-Hempe et al., has a smaller end to the protuberance but requires a stiffening structure. This structure helps form the shape of the protuberance and acts to seal the defect. However, this leaves the cavity partially open and introduces another stiff foreign object into the patient.

Thus, a need exists for a prosthesis of the aforementioned type which is improved compared to those of the prior art, and particularly adapted to allow surgical approaches.

### SUMMARY

According to examples of the invention, a prosthesis formed by a network of biocompatible material having a flat portion and a hollow protuberance projecting from the flat portion. The protrusion has a proximal portion and a distal portion and the distal portion has, in at least one cross-section, a greater area than the proximal portion of the protuberance.

The network of material can be made with monofilament, multifilament polymers, synthetic absorbable or less, such example polypropylene, polyester, polyvinyl fluoride, polylactic acid, polyglycolic acid, polycaprolactone and any copolymers. Such a network or individual filaments can then be coated with polymers, biodegradable or not, providing antibacterial properties or tackiness.

During implantation of the prosthesis, a number of different placement devices or techniques can be used to place the protuberance in the cavity left by the hernia. In one example, a positioning device can be used. The positioning device has a proximal end with a handle and a distal end with a placement tip. One example of a tip includes a circular or partially circular shape having a diameter approximate to approximately 13 mm. Once the flat portion of the prosthesis is placed, the tip can be inserted into the hollow protuberance to extend it in to the cavity. In an alternate example, a balloon is located within the protuberance and first deflated to prevent contact the material to the tissue layers and then inflated by blowing through a syringe, so the distal portion of the protuberance extends in the preperitoneal region, constituting an anchor for the prosthesis.

The distal portion can be shaped as a disc and the proximal portion can be shaped as an hourglass, with a ratio between the height of the distal portion and the height of the proximal portion can be between 1.4 and 3 and with a ratio between the diameter of the distal portion and the diameter of the opening of approximately 1.5 to approximately 2. Further, the difference between the proximal portion may only be slightly higher that of its central part. The flat part may have an oblong shape, or tapering, or a circular shape. Typically, the network and monolithic type and shaped by thermoforming a flat mesh, a portion of which is shaped three-dimensionally so as to form the above-mentioned hollow protuberance, while the remaining portion remains flat.

An example of a prosthesis to treat a hernia, can include a single, monolithic mesh sheet having biocompatible fibers. It further has a flat portion, an unstiffened protuberance formed from the flat portion, and an opening in the flat portion formed by the protuberance and having a first diameter. The protuberance can include a proximal portion closest to the flat portion having a proximal portion height, and a distal portion disposed farthest from the flat portion having a distal portion height, a second diameter, and an end of the distal portion is unstiffened. The proximal portion height can be a distance from the flat portion to a beginning of the distal portion, and the distal portion height can be a distance from the flat portion to the end of the distal portion. Further, the second diameter is greater than the first diameter, and a first ratio of the distal portion height to the proximal portion height is approximately 1.4 to approximately 3.0.

Another example of the prosthesis can have a second ratio of the second diameter to the first diameter, wherein the second ratio can be approximately 1.5 to approximately 2. Also, the second ratio can be approximately 1.5 to approximately 1.8.

An even further example of a prosthesis to treat a hernia, has a single mesh sheet comprising biocompatible fibers, with a flat portion, an unstiffened protuberance formed from the flat portion including a proximal portion closest to the flat portion and a distal portion disposed farthest from the flat portion. An opening can be in the flat portion and formed by the protuberance, it can have a first diameter. The protuberance can narrow from the proximal portion to the distal portion. Also, the distal portion can be rounded. Additionally, the protuberance can have a frustoconical shape.

The invention also includes a method of making a prosthesis to treat a hernia, with the steps of placing a deformed mesh sheet in a mold, wherein the deformed mesh sheet is a single mesh sheet comprising biocompatible fibers having a cylinder formed thereon. Inserting an inflatable mandrel into the cylinder and heating a lower die of the mold to a first temperature. The inflatable mandrel can be inflated with a first pressure to make the cylinder and the lower die contact each other. The lower die can be cooled to a second temperature, lower than the first temperature and the mandrel deflated.

Additional steps include deforming the single mesh sheet, prior to being placed in the mold. This can have the steps of heating the mesh sheet to a third temperature, and applying a first force to the mesh sheet to form the cylinder. Prior to the deforming step there can be a step of weaving the mesh sheet.

The placing step can also include the steps of heating the mold to a fourth temperature, and applying a second force to the mold and mesh sheet. While other steps can be holding the first temperature for a first time before the inflating step or that the inflating step further has a step of maintaining contact between the cylinder and the lower die for a second time before the cooling step.

In at least one example, the first temperature can be approximately 160° C., the second temperature can be approximately 45° C. and the first pressure can be approximately 400 kPa. The third temperature can be approximately 150° C. and the first force can be 50 N and is applied with a second mandrel. The fourth temperature can be approximately 45° C. and the second force can be 10 kgf. The first time can be approximately 30 seconds, and the second time can be approximately 1 minute.

### BRIEF DESCRIPTION OF THE DRAWINGS

This invention is described with particularity in the appended claims. The above and further aspects of this invention may be better understood by referring to the following description in conjunction with the accompanying drawings, in which like numerals indicate like structural elements and features in various figures. The drawings are not necessarily to scale, emphasis instead being placed upon illustrating the principles of the invention.

The drawing figures depict one or more implementations in accord with the present teachings, by way of example only, not by way of limitation. In the figures, like reference numerals refer to the same or similar elements.

FIG. 1 is a top view of a prosthesis of an example of the invention,

FIG. 2 is a side view of the prosthesis of FIG. 1,

FIG. 3 is a sectional view according to the line I-I of FIG. 1,

FIG. 4 is a top view of another prosthesis of an example of the invention,

FIG. 5 is a side view of the prosthesis of FIG. 4,

FIG. 6 top view of a yet further prosthesis of the invention,

FIG. 7 is a side view of the prosthesis of FIG. 6,

FIG. 8 is a top view of still another prosthesis of the invention,

FIG. 9 is a side view of the prosthesis of FIG. 8,

FIGS. 10A and 10B are top a side views, respectively, of examples of the present invention,

FIGS. 11A through 19B are top a side views, respectively, of further examples of the present invention,

FIGS. 20a-20d illustrate an example of the physical formation of the prosthesis of the present invention;

FIG. 21 is a flow chart outlining an example of a method of forming the prosthesis of the present invention;

FIG. 22 is a top, side perspective view of a placement device to use with examples of the prosthesis of the present invention; and

FIG. 23 is an illustration of a prosthesis and placement device in use.

### DETAILED DESCRIPTION

In the following detailed description, numerous specific details are set forth by way of examples in order to provide a thorough understanding of the relevant teachings. However, it should be apparent to those skilled in the art that the present teachings may be practiced without such details. In other instances, well known methods, procedures, components, and/or circuitry have been described at a relatively high-level, without detail, in order to avoid unnecessarily obscuring aspects of the present teachings.

A prosthesis 2 for surgical treatment of indirect and recurrent hernias can be formed by a monolithic mesh of biocompatible material having a flat portion 10 and a protuberance 12 protruding from the flat portion 10. This three-dimensional prosthesis 2 can be obtained through thermoforming a sheet of networked fibers or filaments. The prosthesis 2 may be realized with monofilaments, multifilament synthetic polymers, absorbable polymers, such as polypropylene, polyester, polyvinylidene fluoride, polylactic acid, polyglycolic acid, polycaprolactone and, almost any absorbable or non-absorbable copolymers. The individual filaments can also be coated with other polymers, which can also be biodegradable and confer either antibacterial properties and/or adhesive properties to the mesh.

Other examples of biocompatable absorbable and nonabsorbable materials include, but are not limited to, cotton, linen, silk, polyamides (polyhexamethylene adipamide (nylon 66), polyhexamethylene sebacamide (nylon 610), polycapramide (nylon 6), polydodecanamide (nylon 12) and polyhexamethylene isophthalamide (nylon 61) copolymers and blends thereof), polyesters (e.g. polyethylene terephthalate, polybutyl terphthalate, copolymers and blends thereof), fluoropolymers (e.g. polytetrafluoroethylene) and polyolefins (e.g. polypropylene including isotactic and syndiotactic polypropylene and blends thereof, as well as, blends composed predominately of isotactic or syndiotactic polypropylene blended with heterotactic polypropylene and polyethylene). Suitable absorbable materials include, but are not limited to, homopolymers and copolymers of glycolide, lactide (which includes L-, D-, and meso- forms of lactide and mixtures thereof), [epsilon]-caprolactone, p-dioxanone, trimethylene carbonate, 1,4-dioxepan-2-one, poly(alkylene oxalate), and mixtures of such polymers with each other and with other compatible absorbable compositions as those described.

Note that only certain compositions are strong enough to undergo the heating and stretching noted below to form a monolithic prosthesis. One feature can be that the mesh has a small pore size, and/or high density filament weave. In an example the pore size can be approximately equal to or less than 180 μm. Further examples can have a high tensile strength of at least approximately 120 g/m², or 50 N. In other examples, the monofilament size can be approximately 180 μm, the mesh density of the flat portion 10 prior to the expansion of the protuberance 12 can be approximately 127 g/m². The tensile strength of the mesh can be approximately 7.99 N/mm in one direction and 9.33 N/mm in a perpendicular (90°) direction, while the average pore size prior to expansion can be 698 μm. Further, the mesh should be considered “soft” so as to minimize complications for the patient after implantation.

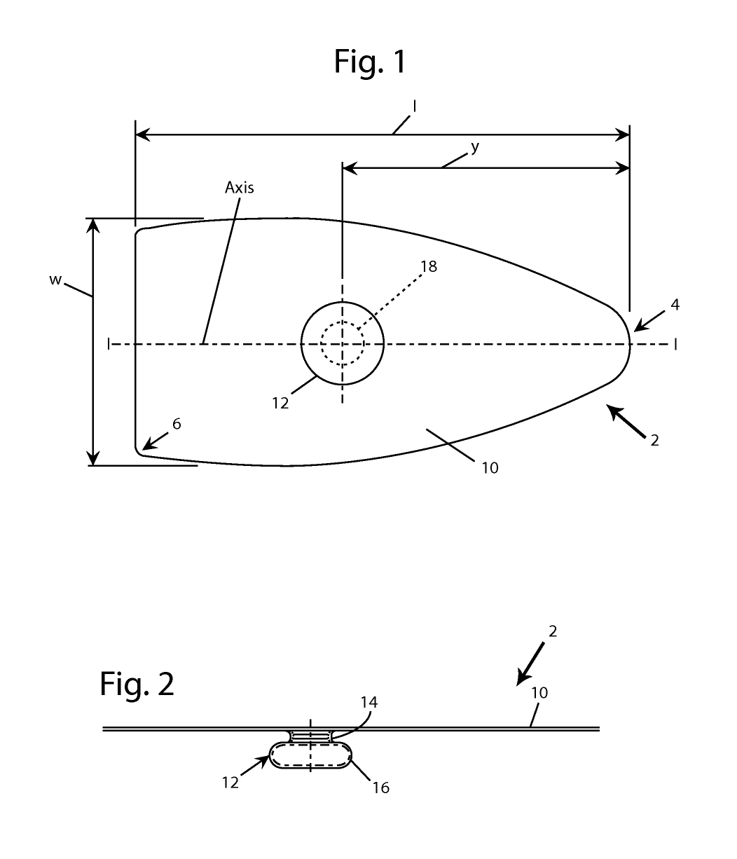

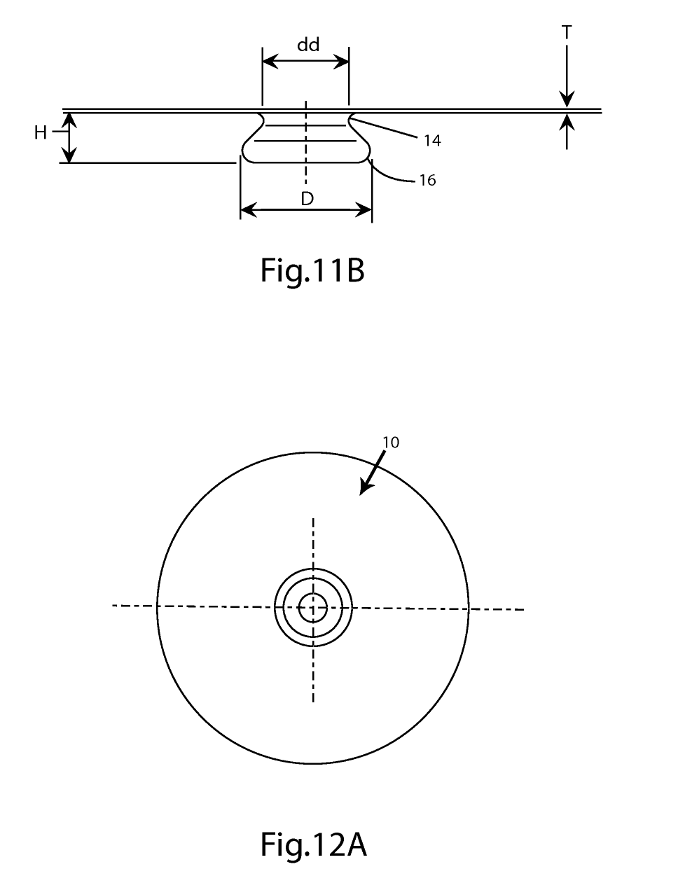

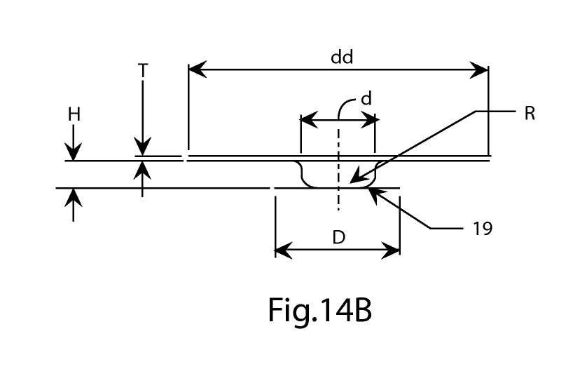

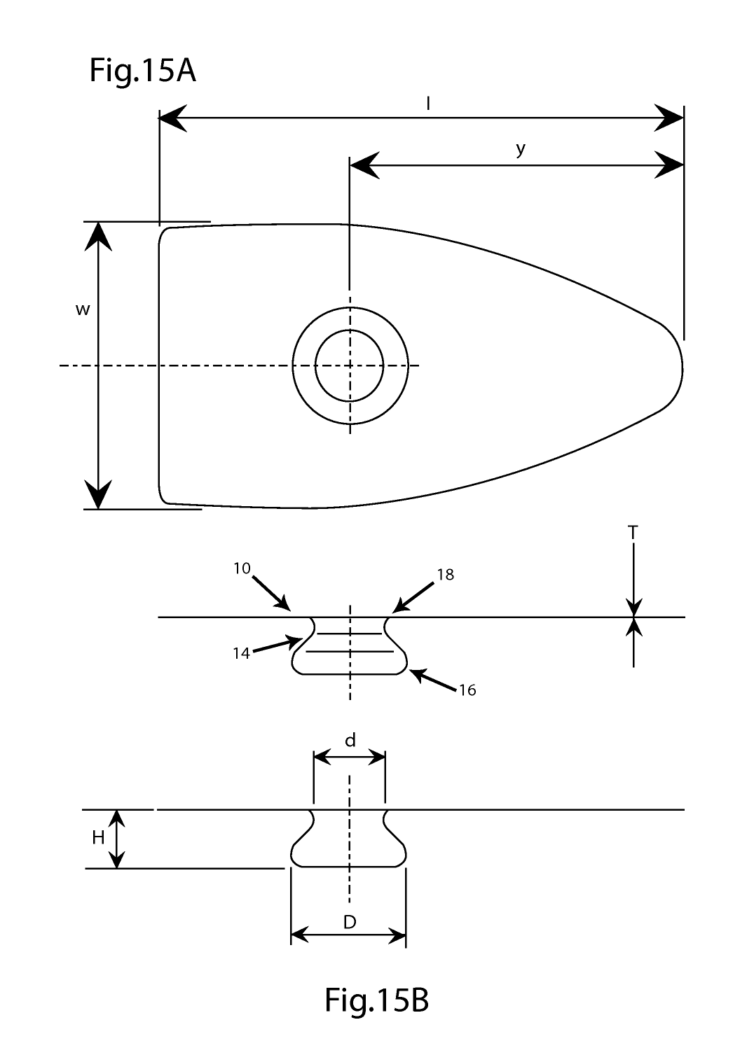

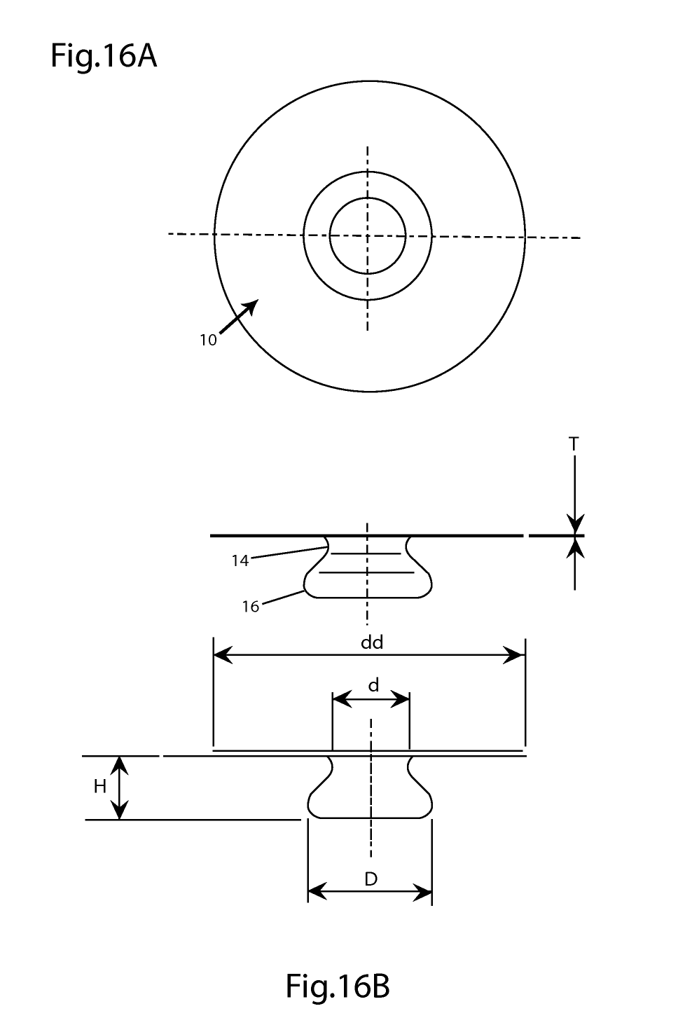

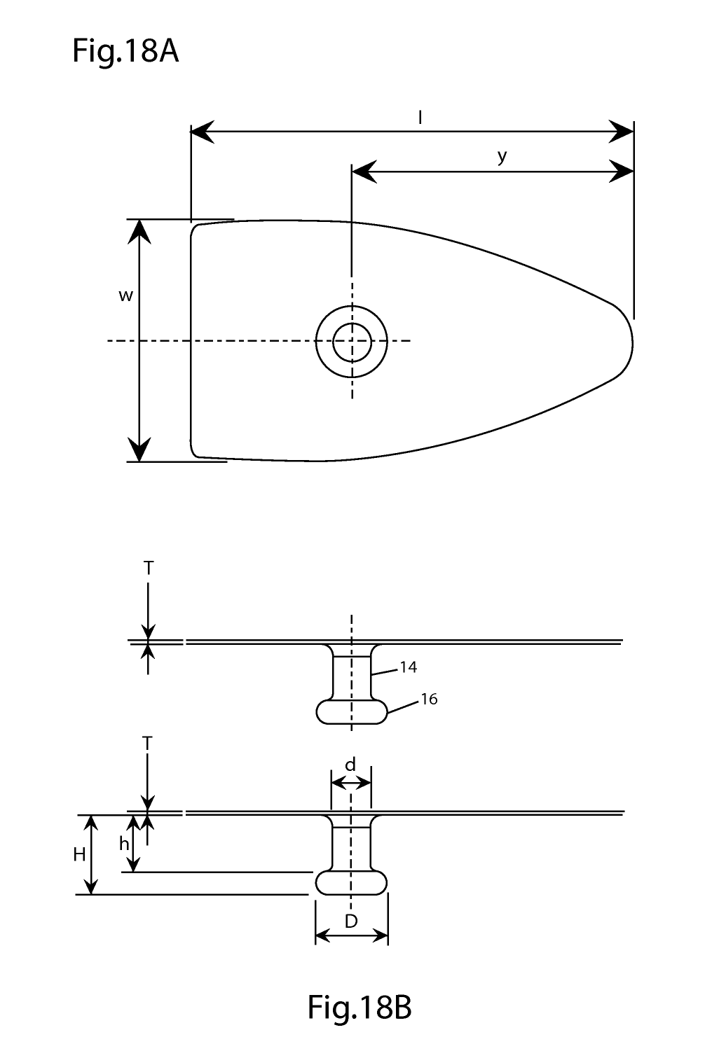

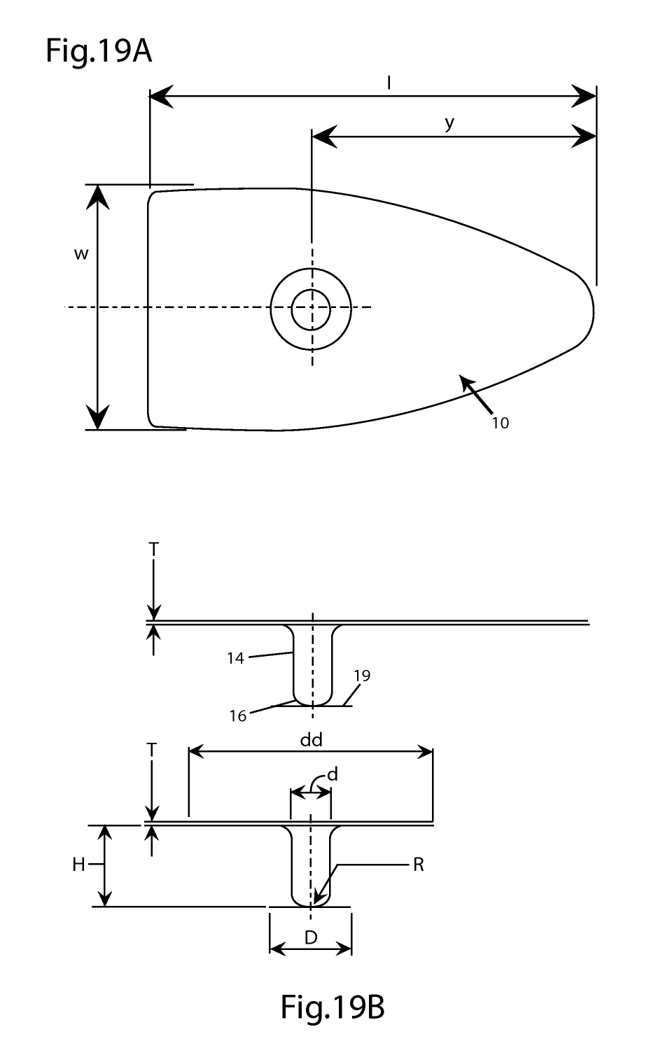

For the structure of the prosthesis 2, the flat portion 10 can have an oblong shape (FIGS. 1, 10A, 11A, 15A, and 17A-19A) or a circular shape (FIGS. 4, 6, 8, 12A-14A and 16A).

In the example where the flat portion 10 is oblong, it can be symmetrical about a longitudinal axis and present a rounded end 4 and an opposite tapered end 6. In one example, a maximum width w can be approximately 60 mm and a maximum length 1 can be approximately 120 mm. The protuberance 12 can be asymmetrically centered along the longitudinal axis of the flat part 10 to a distance y, as taken from the rounded end 4, to a maximum of approximately 70 mm.

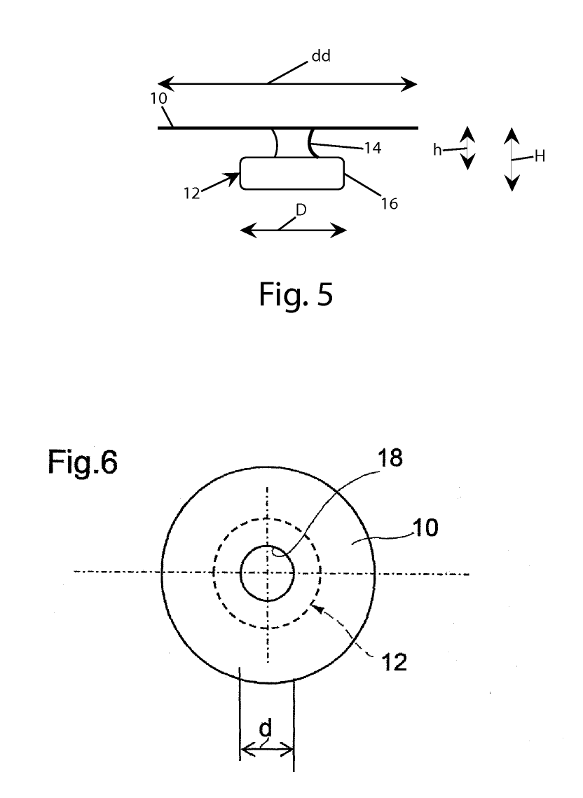

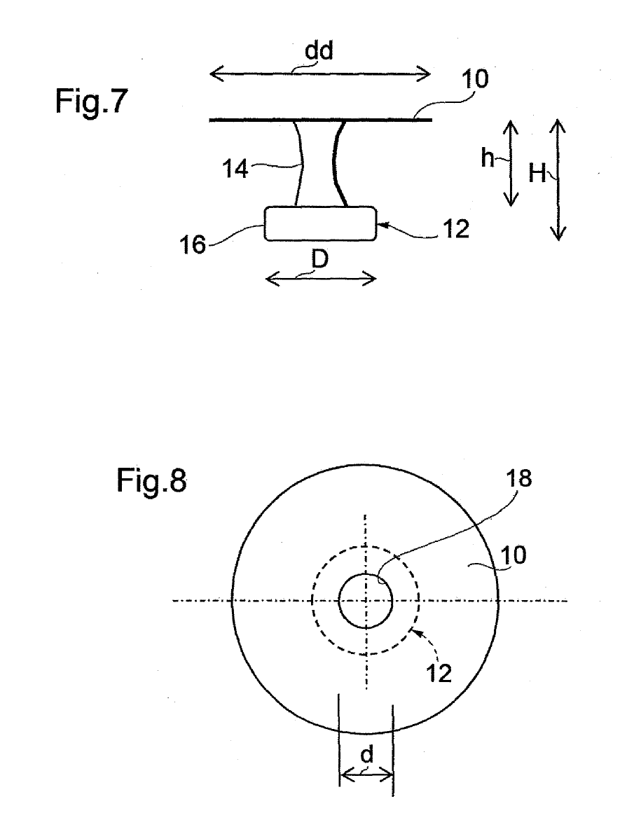

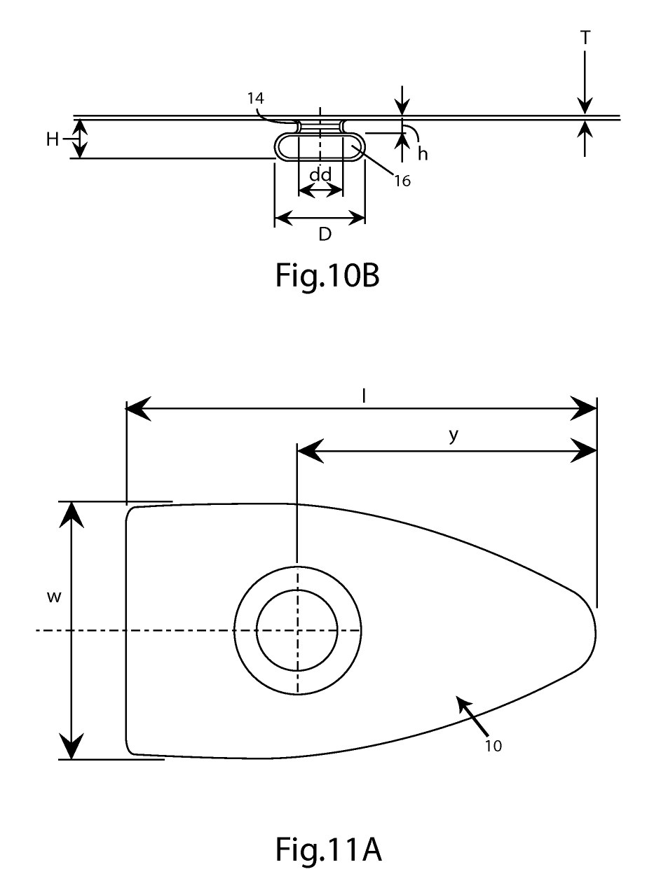

The protuberance 12 can have a proximal portion 14 shaped straight/sloped (FIGS. 10B, 11B, 13B-16B, and 18B-19B) or can be shaped as an hourglass (FIGS. 5, 7, and 9). A distal end 16 can be shaped as a disc with a lateral rounded edge. The proximal portion 14 can have a circular opening of 18 with a diameter d of approximately 10 mm through the flat portion 10. The diameter D of the distal portion 16 can be approximately equal to 20 mm. A height h of the proximal portion 14 can be approximately equal to 3 mm, while an overall height H of the protuberance 12 can be approximately equal to 9 mm.

Another example of the prosthesis 2 for the surgical treatment of recurrent hernias, both direct and indirect, includes a circular shaped flat portion 10. The circular shaped flat portion 10 can have a diameter dd equal to approximately 50 mm. The protuberance 12 again can have a proximal portion 14 shaped straight/sloped or as an hourglass and a distal end 16 can be shaped as a disk. The proximal portion 14 can have the circular opening 18 of diameter d of approximately 10 mm at the flat portion 10. The diameter D of the distal portion 16 can be approximately equal to 20 mm. The height h of the proximal portion 14 can be approximately equal to 5 mm, while the overall height H the protuberance 12 can be approximately equal to 11 mm.

Further examples of the prosthesis 2 for the treatment surgical femoral hernias that have the circular shaped flat portion 10 can have a diameter dd approximately equal to 40 mm. The proximal portion 14 can again be shaped as an hourglass and the distal end 16 is shaped as a disk. The proximal portion 14 has circular opening 18 of diameter d of approximately 10 mm through the flat portion 10.

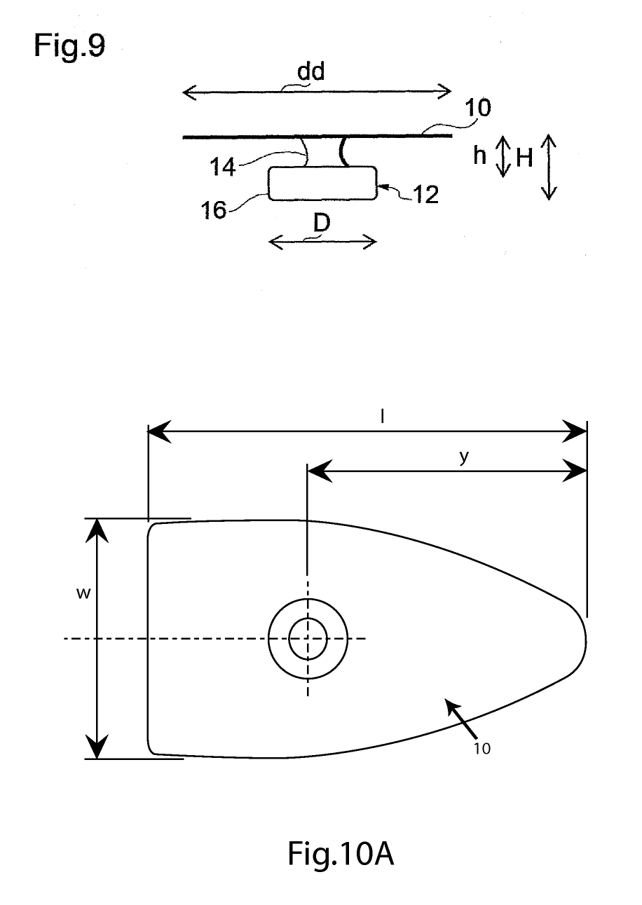

Diameter D of the distal portion 16 can equal approximately 20 mm while the height h of the proximal portion 14 can approximately equal 15 mm. The height H from the distal portion 16 of the protuberance 12 can approximately equal to 21 mm. FIGS. 8 and 9 illustrate another example of the prosthesis 2 of the invention. This prosthesis 2 can be used in laparoscopic techniques and has a circular flat portion 10 with a diameter dd approximately equal to 50 mm. The protuberance 12 has a proximal portion 14 that can be shaped as an hourglass and the distal end 16 can be disk shaped. The proximal portion 14 can have a circular opening 18 having a diameter of approximately 10 mm at the flat portion 10. The diameter D of the distal portion 16 again can be equal to approximately 20 mm. The height h of the proximal portion 14 can be equal to approximately 3 mm, while the full height H the protuberance 12 can be equal to approximately 9 mm.

Other figures illustrate further embodiments of the prosthesis 2. The measurements of the flat portion 10 and the protuberance 12 can vary by example. The length 1 of the ovoid shaped flat portion 10 can range between approximately 110 mm to approximately 120 mm and the width w can be between approximately 60 mm to approximately 80 mm. The diameter dd of circular shaped flat portion 10 can range from approximately 40 mm to approximately 80 mm. Further, the flat portion 10 can have a thickness T which can be approximately 0.6 mm. In another example, the thickness T can be 0.54 mm +/−10%.

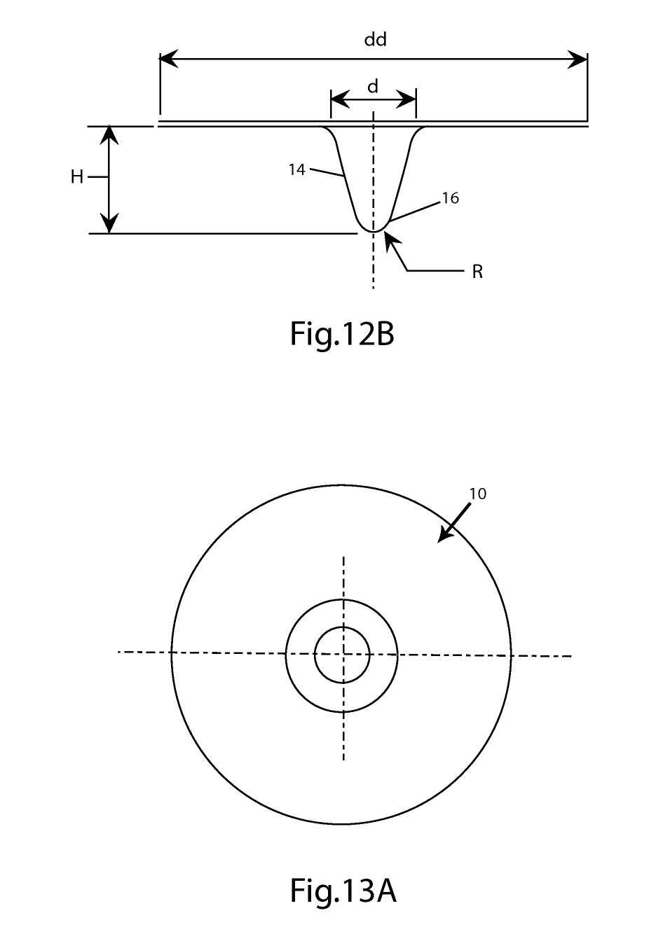

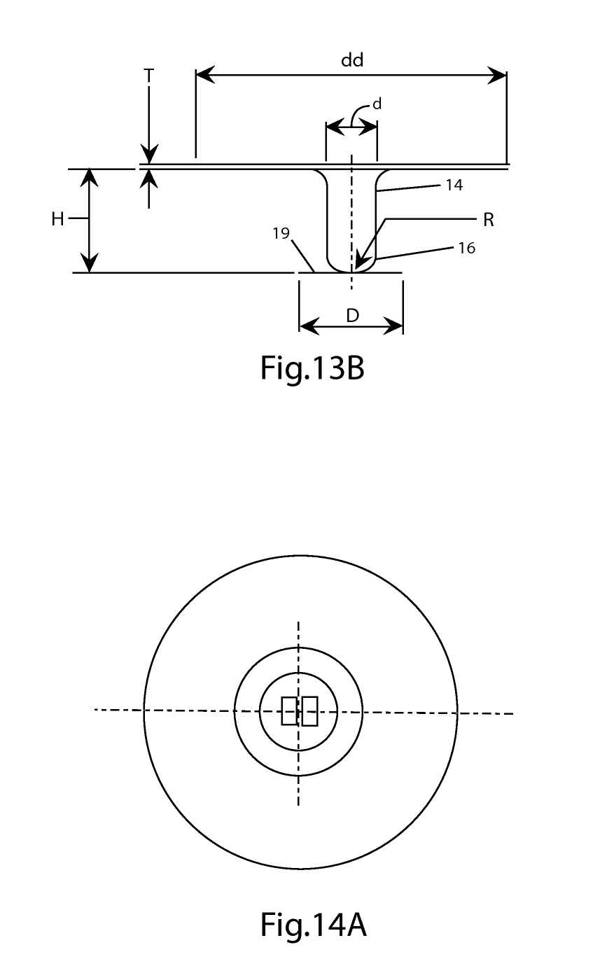

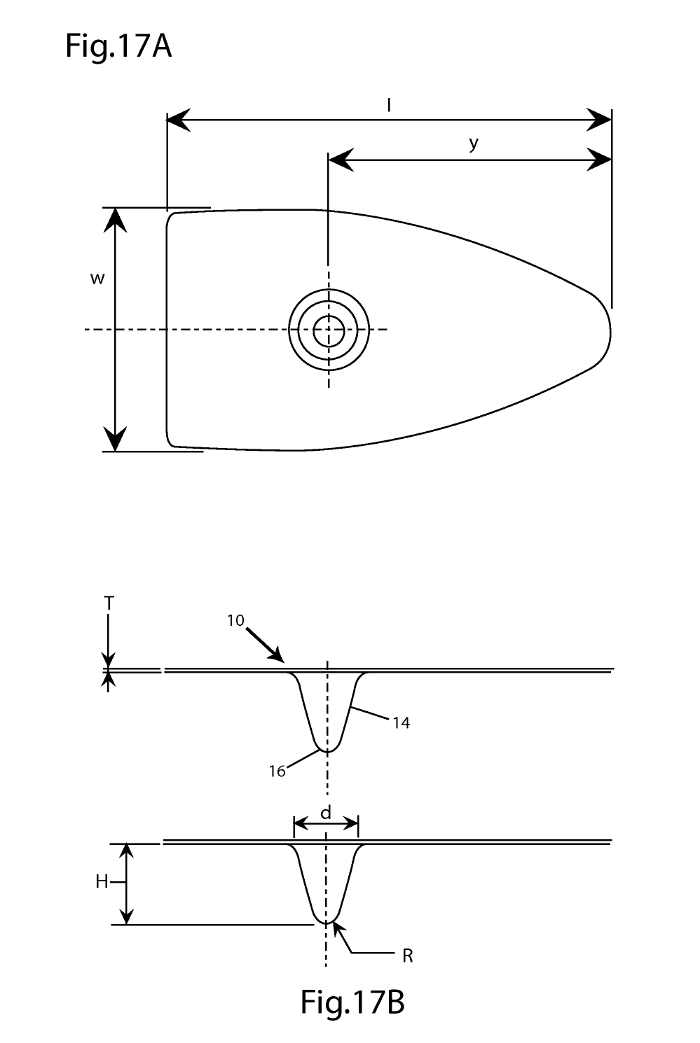

In certain examples the protuberance 12 can have a conical or frustoconical shape with a curved distal end 16. The curved distal ends 16 can have a radius r that can be approximately 3.6 mm. See, FIGS. 12B and 17B. Other examples can have more cylindrical protuberance 12 and a separate mesh 19 can be adhered at the distal portion 16 of the protuberance 12. The separate mesh 19 can be passed through the cavity and acts as the expanded distal portion to anchor the prosthesis 2. See, FIGS. 13B, 14B and 19B.

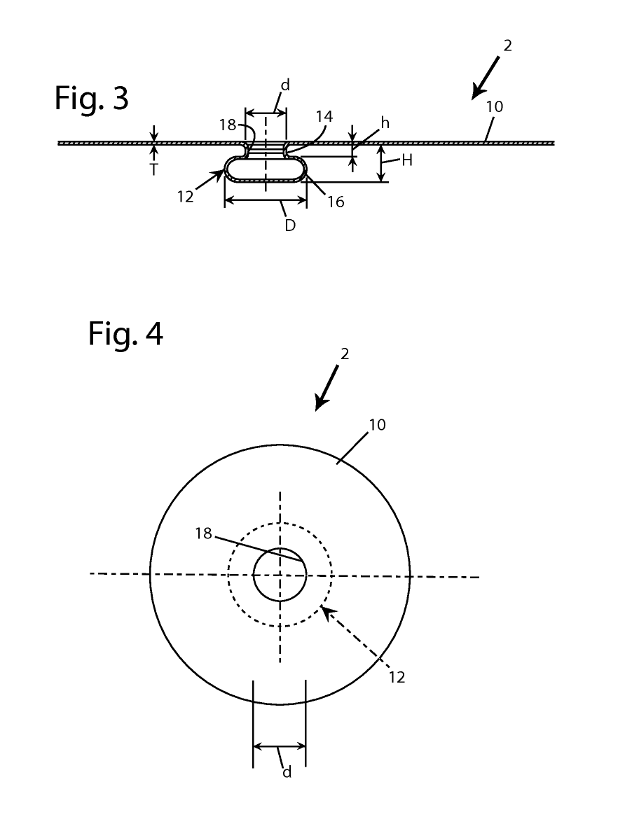

Table 1 below sets out the dimensions for the examples illustrated in FIGS. 1, 3, and 10A-19B. All dimensions are in millimeters.

TABLE 1

| FIG. | l | w | Y | dd | T |
|---|---|---|---|---|---|
| 10 | 110.0 | 60.0 | 70.0 | n/a | 0.6 |
| 11 | 110.0 | 60.0 | 70.0 | n/a | 0.6 |
| 12 | n/a | n/a | n/a | 80.0 | 0.6 |
| 13 | n/a | n/a | n/a | 60.0 | 0.6 |
| 14 | n/a | n/a | n/a | 60.0 | 0.6 |
| 15 | 110.0 | 60.0 | 70.0 | n/a | 0.6 |
| 16 | n/a | n/a | n/a | 60.0 | 0.6 |
| 17 | 110.0 | 60.0 | 70.0 | n/a | 0.6 |
| 18 | 110.0 | 60.0 | 70.0 | n/a | 0.6 |
| 19 | 110.0 | 60.0 | 70.0 | n/a | 0.6 |

Further, there can be relationships between the dimensions that can assist in the use of the prosthetic and Table 2, below, sets out some of those important relationships. Dimensions are in mm.

TABLE 2

| Fig. | d | D | h | H | R | D/d | H/h | R2/d |
|---|---|---|---|---|---|---|---|---|
| 10 | 10.0 | 20.0 | 3.0 | 9.0 | n/a | 2.0 | 3.0 | n/a |
| 11 | 20.0 | 30.0 | 3.5 | 12.0 | n/a | 1.5 | 3.43 | n/a |
| 12 | 16.0 | n/a | n/a | 20.0 | 3.6 | n/a | n/a | 0.5 |
| 13 | 10.0 | 20.0 | n/a | 20.0 | n/a | 2.0 | n/a | n/a |
| 14 | 15.0 | 25.0 | n/a | 6.0 | n/a | 1.7 | n/a | n/a |
| 15 | 15.0 | 25.0 | 3.5 | 12.0 | n/a | 1.7 | 3.43 | n/a |
| 16 | 15.0 | 25.0 | 3.5 | 12.0 | n/a | 1.7 | 3.43 | n/a |
| 17 | 16.0 | n/a | n/a | 20.0 | 3.6 | n/a | n/a | 0.5 |
| 18 | 10.0 | 18.0 | 14.0 | 20.0 | n/a | 1.8 | 1.4 | n/a |
| 19 | 10.0 | 20.0 | n/a | 20.0 | n/a | 2.0 | n/a | n/a |

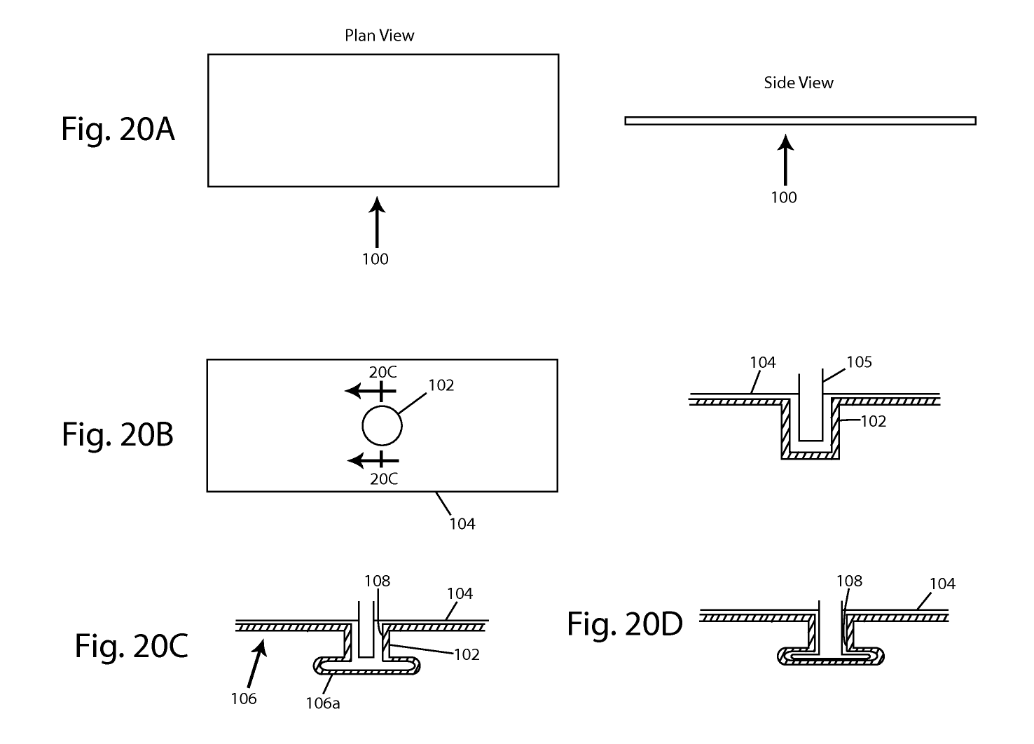

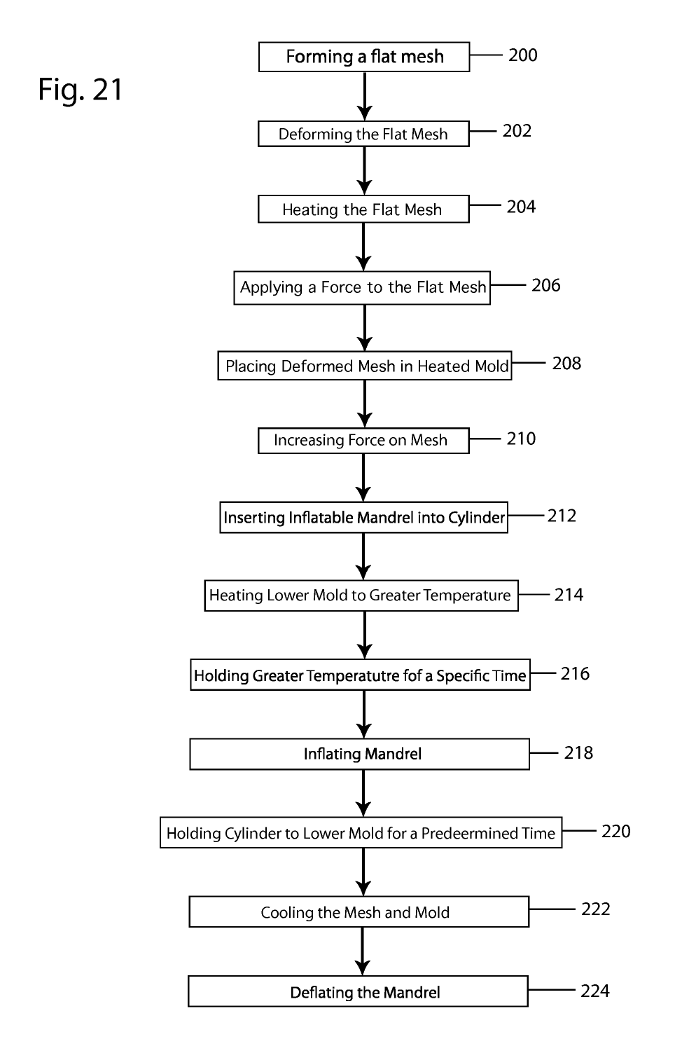

Next we turn to the method of making the prosthesis 2. FIGS. 20A-20D and 21 illustrate examples of the devices and methods described below. A flat mesh 100 can be formed (step 200) using known weaving techniques and the fibers described above. The flat mesh can be deformed (step 202) using a combination of heat (step 204) and force (step 206) to form an approximately uniform cylinder 102. The deformed mesh 104 can then be placed in a heated mold 106 (step 208) under a high force state (step 210). Note that the mold 106 can be shaped as the final shape of at least the distal portion 16 of the protuberance 12, and also can be the final shape of the entire protuberance 12. An inflatable mandrel 108 is inserted into the cylinder (step 212) and the lower part of the mold 106a is heated to a temperate greater than remaining portion of the mold (step 214). The temperature can also be greater than the temperature used in the deforming step (step 204). Once the greater temperature is reached, it can be held for a specific period of time (step 216), and once that time has elapsed, the inflatable mandrel 108 can be inflated (step 218). The mandrel is inflated under high pressure and expands the cylinder 102 until the walls of the cylinder contact the lower mold 106a. The walls of the cylinder can remain in contact with the heated lower mold 106a for a determined amount of time (step 220). Once the determined time has been reached, the mold and mesh can be cooled (step 222). Once a specific temperature is reached, the inflatable mandrel 108 can be deflated (step 224) and the prosthesis 2, now fully formed, can be removed. Note that the prosthesis 2 in this example is fully formed from a single mesh sheet. No other mesh sheets are needed to form the complex shapes illustrated in FIGS. 1-19B. This is a monolithic design.

In a specific example, the flat mesh 100 can either be circular or oblong and is placed in a preforming mold. The preforming mold in this example forms a cylinder 15 mm long and 15 mm in diameter. The mold and mesh are heated to approximately 150° C. and held at that temperature for approximately 1 minute. This softens the fibers of the mesh to allow them to be deformed. A mandrel 105 is then displaced into the mesh sheet at a force of approximately 50 N. The mandrel here is approximately the shape of the cylinder and deforms the fibers to take the shape of the cylinder. The performing mold is opened and the deformed mesh sheet can be transferred to a thermoforming mold. At this stage, in one example, there can be no deliberate cooling of the mesh.

The thermoforming mold can be heated to 45° C. and sealed under a force of approximately 10 kgf. The lower die portion of the thermoforming mold can be any shape and can be the shape of the entire protuberance 12 or just the distal portion 16. In this example, the lower die has a distal section of approximately 25 mm. The lower die portion also receives the cylinder 102 when the deformed mesh is in the thermoforming mold. The inflatable mandrel 108 can be inserted into the cylinder 102 deflated. The lower die can then be heated to 160° C. and held at that temperature for approximately 30 seconds. After the proscribed time, the inflatable mandrel 108 can be inflated using approximately 400 kPa of pressure to force the cylinder 102 to come into contact with the walls of the lower die, thus now taking that shape. The temperature and pressure are held for approximately 1 minute to facilitate the molding of the cylinder into the protuberance 12. After the elapsed time, the pressure is maintained constant but the mold and mesh are air-cooled to a temperature of approximately 45° C., and once the cooled temperature is reached, the mandrel 108 is deflated and the prosthesis 2 has taken its final form.

The example above describes two separate molding devices, but one of ordinary skill in the art can perform the steps on any number of devices, include one device. The steps below can be used on any shape or size flat part 10 to form any size or shape protuberance 12 to any of the above disclosed ratios. Further, in one example, the inflatable mandrel can be made from silicon. Furthermore, in one example the entire process from when the completed mold is preformed till it completes thermoforming, can be a maximum of 15 minutes and a minimum of 5 minutes.

To surgically implant the prosthesis 2, the surgeon begins to place the flat portion 10 over the hernia. The surgeon can use a finger or other tool to move the protuberance 12 into the opening and have the distal portion 16 pass through. For example, an expandable balloon can be placed within the protuberance 12 that is inserted in the opening left by the reduction in muscle of the hernia. The balloon can then be inflated to a volume equal to 5 cm³, so that the distal portion 16 of the protuberance 12 is placed in the preperitoneal region.

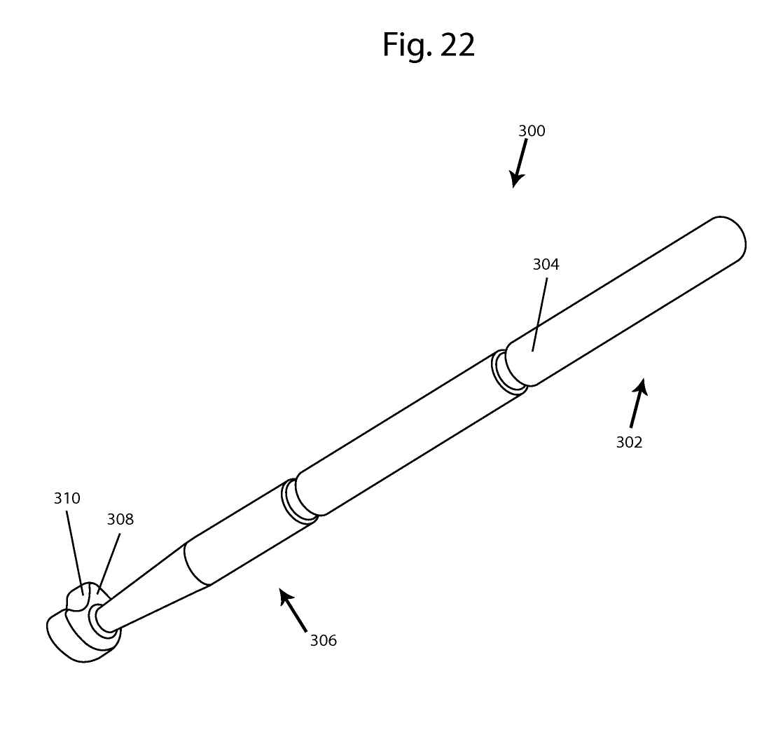

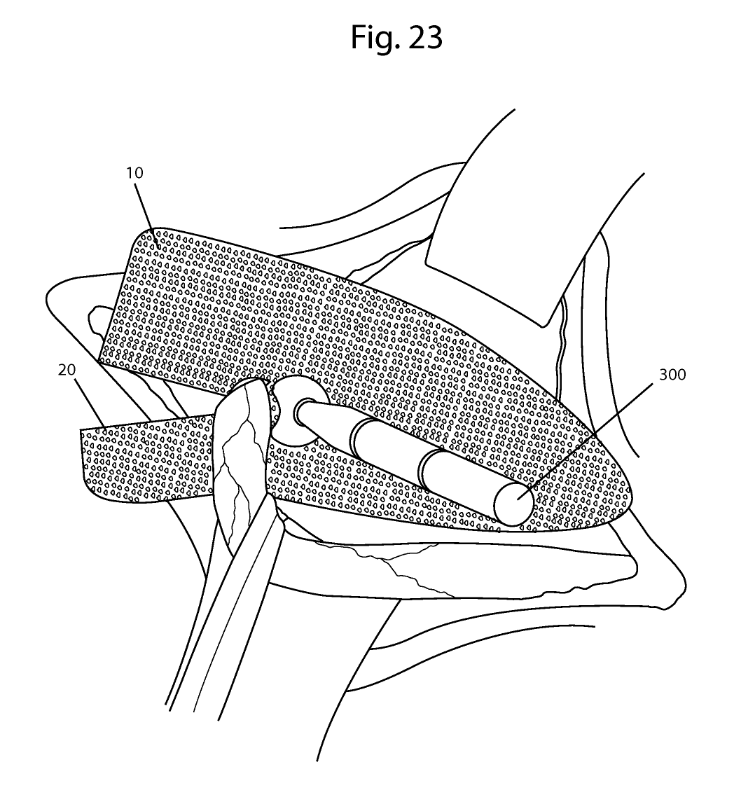

In another example, illustrated in FIGS. 22 and 23, during implantation of the prosthesis 2 a positioning device 300 can be used to place the protuberance 12 in a cavity left by the hernia. The positioning device 300 has a proximal end 302 with a handle 304 and a distal end 306 with a placement tip 308. One example of the tip 308 includes a circular or partially circular shape having a diameter approximate to 13 mm. Another example of the tip 308 has a semi-circular cut-out 310 that helps with avoiding damage to the spermatic cord. Once the flat portion 10 of the prosthesis 2 is placed, the tip 308 can be inserted into the hollow protuberance 12 to extend it in to the cavity.

For a further example, the size and the shape of the positioning device 300 can be important. Regardless of the ultimate shape of the tip 308, it should not be sharp or have shape edges. Thus, the tip 308 can have rounded corners. Another important feature of one example is that the tip 308 (and by extension, the handle 304) be smaller than circular opening 18 (with the diameter d) in the prosthesis 2. Examples of the diameter d are noted above. During placement, the surgeon moves the tool inside the cavity in order to position the protuberance 12 and extend out the distal portion 16. FIG. 23 also illustrates that the flat portion 10 of any of the prosthesis 2 may also be notched 20 (i.e. with a key-hole, semi-circular notch, or other notch shape) to facilitate the passage of the spermatic cord when the device is used in indirect hernia. FIG. 23 also illustrates the positioning device 300 in use.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

What is claimed is:

1. A prosthesis to treat a hernia, comprising:
a single mesh sheet comprising biocompatible fibers, further comprising:
a flat portion;
an unstiffened protuberance formed from the flat portion; and
an opening in the flat portion formed by the protuberance and having a first diameter;
wherein the protuberance comprises:
a proximal portion closest to the flat portion having a proximal portion height; and
a distal portion disposed farthest from the flat portion having a distal portion height, a second diameter and an end of the distal portion is unstiffened,
wherein the proximal portion height is a distance from the flat portion to a beginning of the distal portion,
wherein the distal portion height is a distance from the flat portion to the end of the distal portion,
wherein the second diameter is greater than the first diameter, and
wherein a first ratio of the distal portion height to the proximal portion height is approximately 1.4 to approximately 3.0.

2. The prosthesis of claim 1, further comprising a second ratio of the second diameter to the first diameter, wherein the second ratio is approximately 1.5 to approximately 2.

3. The prosthesis of claim 2, wherein the second ratio is approximately 1.5 to approximately 1.8.

4. A prosthesis to treat a hernia, comprising:
a single mesh sheet comprising biocompatible fibers, further comprising:
a flat portion;
an unstiffened protuberance formed from the flat portion comprising a proximal portion closest to the flat portion and a distal portion disposed farthest from the flat portion; and
an opening in the flat portion formed by the protuberance and having a first diameter;
wherein the protuberance narrows from the proximal portion to the distal portion.

5. The prosthesis of claim 4, wherein the distal portion is rounded.

6. The prosthesis of claim 4, wherein the protuberance comprises a frustoconical shape.

7. A method of making a prosthesis to treat a hernia, comprising the steps of:
placing a deformed mesh sheet in a mold, wherein the deformed mesh sheet is a single mesh sheet comprising biocompatible fibers having a cylinder formed thereon;
inserting an inflatable mandrel into the cylinder;
heating a lower die of the mold to a first temperature;
inflating the inflatable mandrel with a first pressure to make contact the cylinder and the lower die;
cooling the lower die to a second temperature, lower than the first temperature; and
deflating the inflatable mandrel.

8. The method of claim 7, further comprising the steps of:
deforming the single mesh sheet, comprising the steps of:
heating the mesh sheet to a third temperature; and
applying a first force to the mesh sheet to form the cylinder.

9. The method of claim 8, further comprising the step of weaving the mesh sheet prior to the deforming step.

10. The method of claim 7, wherein the placing step further comprises the steps of:
heating the mold to a fourth temperature; and
applying a second force to the mold and mesh sheet.

11. The method of claim 7, further comprising the step of holding the first temperature for a first time before the inflating step.

12. The method of claim 7, wherein the inflating step further comprises the step of maintaining contact between the cylinder and the lower die for a second time before the cooling step.

13. The method of claim 7, wherein the first temperature is approximately 160° C., the second temperature is approximately 45° C. and the first pressure is approximately 400 kPa.

14. The method of claim 8, wherein the third temperature is approximately 150° C. and the first force is 50 N and is applied with a second mandrel.

15. The method of claim 10, wherein the fourth temperature is approximately 45° C. and the second force is 10 kgf.

16. The method of claim 11, wherein the first time is approximately 30 seconds.

17. The method of claim 12, wherein the second time is approximately 1 minute.
